(19) United States
(12) Patent Application Publication
(10) Pub. No.: US 2006/0030778 A1
(43) Pub. Date: Feb. 9, 2006

(54) Ultrasonic transmission films and devices for hygienic transducer surfaces

(76) Inventors: John D. Mendlein, Scituate, MA (US); Philipp Lang, Lexington, MA (US)

Correspondence Address: WILMER CUTLER PICKERING HALE AND DORR LLP, 60 STATE STREET, BOSTON, MA, 02109, US

(21) Appl. No.: 11/191,804

(22) Filed: Jul. 28, 2005

Related U.S. Application Data
(63) Continuation of application No. 10/436,824, filed on May 13, 2003, now abandoned.
Application No. 10/436,824 is a division of application No. 09/314,206, filed on May 19, 1999, now abandoned.
Application No. 09/314,206 is a continuation of application No. PCT/US98/17242, filed on Aug. 19, 1998.
Application No. 09/314,206 is a continuation-in-part of application No. 09/096,857, filed on Jun. 12, 1998, now abandoned.
Application No. 09/314,206 is a continuation-in-part of application No. 08/914,527, filed on Aug. 19, 1997, now abandoned.

(51) Int. Cl. A61B 8/14; A61B 8/00; A61B 8/12

(52) U.S. Cl. 600/437

(57) ABSTRACT

The present invention provides for methods and devices for reducing medical probe contamination by providing rigid probe holders.

### TECHNICAL FIELD

This application claims the benefit of all rights accorded under Sections 120 and 119 with respect to the following applications and is a continuation of PCT application serial no. PCT/US98/17242, filed on Aug. 19, 1998 by Mendlein and Lang, a continuation-in-part application of U.S. application Ser. No. 09/096,857, filed on Jun. 12, 1998 by Mendlein and Lang and a continuation-in-part application of U.S. application Ser. No. 08/914,527, filed on Aug. 19, 1997, by Lang and Mendlein, which are all incorporated herein by reference.

### TECHNICAL FIELD

The invention relates to holders for probes using waveform energy, particularly ultrasonic probes for medical applications.

### INTRODUCTION

For over thirty years ultrasound has been used as a safe and effective diagnostic tool. During this time, many different types of ultrasound methods and devices have been developed, such as imaging techniques, Doppler flow methods, and speed of sound measurements, as well as their respective devices. Clinicians use such methods and devices in a variety of clinical settings that range from obstetrics to cardiology.

Imaging methods and devices can provide details of the topography of various tissues. Ultrasound imaging is extremely cost effective and easy to operate by comparison. For many imaging situations, ultrasound is often preferred over magnetic resonance imaging for patient management because ultrasound imaging provides relatively fast imaging times and sufficient interrogation of anatomic details using comparatively inexpensive devices and operation costs.

Doppler flow methods and devices can provide information about blood flow in tissues. Doppler systems have been used from many years to inexpensively monitor blow flow in the vessels of the body. Doppler systems can also be combined with imaging techniques to probe additional details of vessel function, such as velocity profiles across the vessel.

Because ultrasound techniques have been extensively used for many years, the side effects of ultrasound are not an issue for clinicians. The safety of ultrasound is well recognized in the field of medical imaging and diagnostics. As Bushberg et al points out:
“Ultrasound has established a remarkable safety record related to potential bioeffects caused by the exposure to mechanical radiation used at the typical intensity levels for diagnostic imaging and Doppler exams. In fact, there has never been any confirmed bioeffects on either patients or operators of diagnostic ultrasound procedures.” The Essential Physics of Medical Imaging, Bushberg, J. T., et al, Chapter 12, page 414 (1994).

Despite the widespread use of ultrasound as a safe and effective diagnostic tool many types of ultrasound technology have not been developed or clinical applications of existing ultrasound technology have not been recognized. Many areas remain unexplored and the inventors of the present invention offer new technologies that are particularly applicable to ultrasonic diagnostic, as well as other medical and non-medical applications.

Although ultrasound is a safe technique that has been used for many years, ultrasound technologies have not intensely focused on reducing probe contamination or inter-patient transference of pathogens or other contact transmittable diseases related to probe contact. In the general population, there has been an increase in incidence and rate of transmission of sexually transmitted diseases (STDs), including acquired immunodeficiency syndrome (AIDS). There has also been a rise in the development of increasingly antibiotic-resistant strains of disease-causing organisms and drug resistant pathogens, such as those responsible for diseases such as syphilis and gonorrhea and other replicating pathogens. While such increases in transmittable pathogens have been observed, attention to decreasing transmission of diseases in diagnostic interrogation or therapeutic settings using medical probes with patients has been wanting. For instance, the technologies for reducing disease transmission during ultrasound interrogation of integument covered structures, such as the abdomen, testicles, thyroid, face, and feet have not been thoroughly addressed.

Consequently, the present inventors have recognized the need, among other things, to provide reliable, inexpensive and convenient devices and methods for such applications, particularly for reducing probe contamination. The methods and devices provided herein enable easy to use and cost effective devices and methods for reducing probe contamination while providing accurate and more reproducible interrogation of patients with medical probes.

TABLE OF CONTENTS

TECHNICAL FIELD
BACKGROUND
SUMMARY
BRIEF DESCRIPTION OF FIGURES
DETAILED DESCRIPTION OF THE INVENTION

1.0 ABBREVIATIONS AND DEFINITIONS
2.0 INTRODUCTION
3.0 HOLDERS AND FILMS

Introduction
Holders and Holders with Sonolucent Films
An Example of a Holder
Holders with Predetermined, Maintained shapes
Removable Holders
Molded Holders
An Example of A Universal Holder
An Example of An Ultrasound Holder

4.0 STACKS AND DISPENSING HOLDERS

Stacks and Advantages of Stacks
Holder Dispensing Stations

5.0 DEVICES FOR MANUFACTURE AND METHODS

Application Sites
Application to Medical Treatments

6.0 THERAPEUTIC KITS

Kits
Use in Medical Conditions and Treatments

EXAMPLES

GENERAL MATERIALS AND METHODS
EXAMPLE 1: ULTRASONOGRAPHIC MEASUREMENT USING
POLYMER FILMS

PUBLICATIONS

U.S. PATENT DOCUMENTS
FOREIGN PATENT DOCUMENTS
OTHER PUBLICATIONS

CLAIMS
ABSTRACT

### SUMMARY

The inventors of the present invention recognized, among other things, a need in the ultrasound field for transmission films and holders for transducers, particularly for transducers adapted to interrogate the external surface of an object or a subject. Transmission films and holders of the invention can provide for hygienic probes of tissues, including the genitalia where transmission of sexually transmitted diseases (“STDs”) may occur. Such transmission films and holders can offer many advantages including, reduced transmission of STDs and other contact transmitted diseases, low cost of manufacture, enhanced or more reproducible recording, convenience and hygiene of disposable articles, and reduced operator error in maintaining inter-patient hygiene. The inventors also discovered a need for dispensing such transmission films and holders in a convenient, user and manufacturing friendly manner.

The invention includes a device that comprises a holder for an ultrasonic probe that is adapted for skin-interrogation of tissues subjacent to a skin interrogation site. The holder is adapted to fit at least a portion of the interrogation surface of the ultrasonic probe. The interrogation surface of the probe is a portion of the probe designed to transmit or receive ultrasonic waves. The holder typically includes 1) a securing portion for securing the holder to the ultrasonic probe and 2) an interrogation window in acoustic alignment with at least a section of the interrogation surface. A sonolucent film may be included to cover the interrogation window.

The holder is typically made of a hard, polymeric material. The holder is usually designed to be flexible while maintaining the general shape of the probe for which it was designed to fit. The holder may have flexible extensions or flanges that secure the holder to the probe. The holder may be constructed from a rigid, flexible plastic that can bend slightly as the probe is inserted in the holder. Once the probe is inserted in the holder, the holder will grasp the probe as the flexible plastic will be tensioned on the probe. The polymeric material of the holder often is a rigid, injection molded polymer, which is easy to manufacture on a large scale. The holder is usually designed with a region(s) having a cross sectional thickness greater than the sonolucent film's cross sectional thickness. The sonolucent film may include an applied gel on the sonolucent film's exterior interrogation-side (i.e. the side in contact with the patient) to enhance acoustic communication. The sonolucent film may also include an applied gel on the sonolucent film's interior interrogation-side to enhance acoustic communication.

The invention includes a device that comprises a rigid, plastic holder for a probe, wherein the holder is of a generally predetermined shape. The holder also typically has generally preset three-dimensional dimensions that are maintained without the insertion of the probe, such as an ultrasound source or detector. The rigid, plastic holder comprises an interrogation region for interrogation of an exterior interrogation surface of an object or patient. The interrogation region can be dimensioned to snugly fit over a housing or frame for the probe's electromagnetic or ultrasound source or detector while permitting interrogation through the interrogation region.

A rigid holder for a probe offers the advantage of holding the shape of the probe without the probe being introduced into the holder. A rigid holder can also allow for rapid engagement of the probe with the holder and easy removal (with one hand). Rigid holders may also be stacked for quick and reliable deployment, as described herein. In addition, the holders can include a predetermined amount of transmission enhancement fluid or layer that increases the reproducibility of interrogation using probes that can be used with transmission enhancement fluids.

The invention also provides for a device comprising a stack of holders for a probe. Each holder comprises an exterior region and an interior region. The exterior region of each holder is adapted to fit into the interior region of the next holder in the stack. Alternatively, the interior region of each holder is adapted to fit into the exterior region of the next holder in the stack. As another alternative, the exterior region of each holder is adapted to fit into the interior region of the next holder in the stack and the interior region each holder is adapted to fit into the exterior region of the next holder in the stack.

Stacks of the invention offer a number of advantages, including 1) one handed donning of holders on to probes, 2) convenient maintenance of the hygiene or sterility of holders, 3) convenient storage of holders, and 4) easy repetitive donning of holders on to probes for rapid multiple interrogations.

The invention includes methods and devices for manufacturing and testing articles of the invention. Such methods and devices can also be used for manufacturing and testing many other types of objects, particular objects that can have a structural feature interrogated by ultrasonic methods.

The invention also includes a therapeutic kits.

### BRIEF DESCRIPTION OF THE FIGURES

FIG. 1 A-B show examples of a holder of the invention in a front and cross sectional view, respectively.

FIG. 1C-D show examples of acoustic coupling gel layers applied to holders of the invention.

FIG. 2 A shows a front view of a holder of the invention with a probe.

FIG. 2 B shows a front view of a holder of the invention with a probe with a securing collar.

FIG. 2 C shows a front view of a holder of the invention with a probe with a securing collar.

FIG. 2 D shows a front view of a holder of the invention with a probe with an acoustic coupling gel applicator and reservoir.

FIG. 2 E shows a side view of a holder of the invention with a probe with an acoustic coupling gel applicator and reservoir.

FIGS. 3A and B show embodiments of the invention comprising an ultrasound transducer secured to a subject or a tissue surface with an adhesive probe holder, which is preferably used for intermittent or continuous recording.

FIG. 4 shows one embodiment of the invention comprising an ultrasound transducer attached to a separate positioning frame with an attachment member.

FIG. 5A shows an example of a stack of holders in a rack in cross sectional view.

FIG. 5B shows an example of a stack of holders with caps and gel layers in a rack in cross sectional view.

FIG. 5C shows an example of a stack of holders with caps and gel layers in a rack that elevates the rack so that a holder is accessible from the top of the rack in cross sectional view.

FIG. 6A shows an example of a manufacturing process of the invention as a flow chart.

FIG. 6B shows an example of a manufacturing process of the invention as a flow chart.

FIG. 7 shows an example of a manufacturing or testing device of the invention for dispensing a transmission enhancing fluid or testing a surface.

### DETAILED DESCRIPTION OF THE INVENTION

1.0 Abbreviations and Definitions

ABBREVIATIONS include BUA (broad band ultrasound attenuation), and SOS (speed of sound).

Acoustic communication refers to the passage of ultrasound waves between two points in a predetermined manner. Usually, this is accomplished by selecting a desired pathway between the two points that permits the passage of ultrasound waves either directly or indirectly. Direct passage of ultrasound waves would occur, for instance, when an ultrasound crystal is directly disposed to (usually touching) an acoustic coupling material, such as a composite. Indirect passage of ultrasound waves would occur, for instance, when an ultrasound crystal is located at a predetermined distance from an acoustic coupling material or when a number of acoustic coupling materials, often heterogeneous materials, form two or more layers.

Acoustic coupler refers to a connection or plurality of connections between an ultrasound crystal and a substance that reflects or passes ultrasound pulses and is not part of the device. The acoustic coupler will permit passage of ultrasound waves. It is desirable for such couplers to minimize attenuation of ultrasound pulses or signals and to minimize changes in the physical properties of an ultrasound wave, such as wave amplitude, frequency, shape and wavelength. Typically, an ultrasound coupler will either comprise a liquid, gel or other substantially soft material, such as a pliable polymer matrix, that can transmit ultrasound pulses. Alternatively, an ultrasound sound coupler can be a substantially solid material, such as a polymer matrix, that can transmit ultrasound pulses. An ultrasound coupler is usually selected based on its acoustic impedance match between the object being interrogated and the ultrasound crystal(s). If a reflective surface is desired, for instance as a spatial marker, a larger impedance difference is selected compared to situations where it is advantageous to minimize a reflective surface to avoid a sharp reflective surface.

Acoustic coupling material is a material that passes ultrasound waves, usually from a probe to a subject or tissue to be interrogated. It is usually not a living material and is most often a polymer or gel.

Anatomical region refers to a site on the surface of the skin, tumor, organ or other definable biomass that can be identified by an anatomical feature or location. Usually, such a region will be definable according to standard medical reference methodology, such as that found in Williams et al., Gray's Anatomy, 1980.

A-scan refers to an ultrasound technique where an ultrasound source transmits an ultrasound wave into an object, such as patient's body, and the amplitude of the returning echoes (signals) are recorded as a function of time. Only structures that lie along the direction of propagation are interrogated. As echoes return from interfaces within the object or tissue, the transducer crystal produces a voltage that is proportional to the echo intensity. The sequence of signal acquisition and processing of A-scan data in a modern ultrasound instrument usually occurs in six major steps:
Detection of the echo (signal) occurs via mechanical deformation of the piezoelectric crystal and is converted to an electric signal having a small voltage.
Pre-amplification of the electronic signal from the crystal, into a more useful range of voltages is usually necessary to ensure appropriate signal processing.
Time Gain Compensation compensates for the attenuation of the ultrasound signal with time, which arises from travel distance. Time gain compensation may be user-adjustable and may be changed to meet the needs of the specific application. Usually, the ideal time gain compensation curve corrects the signal for the depth of the reflective boundary. Time gain compensation works by increasing the amplification factor of the signal as a function of time after the ultrasound pulse has been emitted. Thus, reflective boundaries having equal abilities to reflect ultrasound waves will have equal ultrasound signals, regardless of the depth of the boundary.
Compression of the time compensated signal can be accomplished using logarithmic amplification to reduce the large dynamic range (range of smallest to largest signals) of the echo amplitudes. Small signals are made larger and large signals are made smaller. This step provides a convenient scale for display of the amplitude variations on the limited gray scale range of a monitor.
Rectification, demodulation and envelope detection of the high frequency electronic signal permits the sampling and digitization of the echo amplitude free of variations induced by the sinusoidal nature of the waveform.
Rejection level adjustment sets the threshold of signal amplitudes that are permitted to enter a data storage, processing or display system. Rejection of lower signal amplitudes reduces noise levels from scattered ultrasound signals.

B-scan refers to an ultrasound technique where the amplitude of the detected returning echo is recorded as a function of the transmission time, the relative location of the detector in the probe and the signal amplitude. This is often represented by the brightness of a visual element, such as a pixel, in a two-dimensional image. The position of the pixel along the y-axis represents the depth, i.e. half the time for the echo to return to the transducer (for one half of the distance traveled). The position along the x-axis represents the location of the returning echoes relative to the long axis of the transducer, i.e. the location of the pixel either in a superoinferior or mediolateral direction or a combination of both. The display of multiple adjacent scan lines creates a composite two-dimensional image that portrays the general contour of internal organs.

Chip refers to any current and future electronic compact hardware device within a computational unit that can be used as an aid in controlling the components of an ultrasound unit including: 1) timing and synchronizing trigger pulses and subsequent transmission of ultrasound waves, 2) measuring and analyzing incoming ultrasound signals, 3) instructing dispensing of acoustic coupling fluid, 4) instructions for testing surfaces ultrasonically, 5) instructing a transfer system to transfer articles of manufacture, 6) comparing data to predetermined standards and data cut-offs (e.g. electronic filtering), 7) generating anatomical maps of ultrasound parameters, and 8) performing multiple other simple and complex calculations. Chips are preferably integrated circuits, usually etched-silicon circuits, of micron dimension or less.

Computational unit refers to any current or future software, integrated circuit, chip or other device used for calculations, such as ultrasonic calculations, now developed or developed in the future. The computational unit may be designed to control the ultrasound generator or source, for defining or varying the firing rate and pulse repetition rate (as well as other parameters related to the ultrasound generator or source), for measuring the reflected signal, for image reconstruction in B-scan mode and for filtering and thresholding of the ultrasound signal. Other applications of the computational unit to the methods and devices described herein will be recognized by those skilled in the art. The computational unit may be used for any other application related to this technology that may be facilitated with use of computer software or hardware.

Crystal refers to the material used in the ultrasound transducer to transmit ultrasound waves and includes any current and future material used for this purpose. Crystals typically consist of lead zirconate titanate, barium lead titanate, lead metaniobate, lithium sulfate and polyvinylidene fluoride or a combination thereof. A crystal is typically a piezoelectric material, but any material that will contract and expand when an external voltage is applied can be used, if such a material can generate ultrasound waves described herein and known in the art. Crystals emit ultrasound waves because the rapid mechanical contraction and expansion of the material moves the medium to generate ultrasound waves. Conversely, when incoming ultrasound waves deform the crystal, a current is induced in the material. The materials then emit an electrical discharge that can be measured and, ultimately, with B-scan technology can be used to reconstruct an image. Crystals or combinations of crystals with dipoles that approximate the acoustic impedance of human tissue are preferred, so as to reduce the impedance mismatch at the tissue/probe interface.

C-scan refers to an ultrasound technique where additional gating electronics are incorporated into a B-scan to eliminate interference from underlying or overlying structures by scanning at a constant-depth. An interface reflects part of the ultrasound beam energy. All interfaces along the scan line may contribute to the measurement. The gating electronics of the C-mode rejects all returning echoes except those received during a specified time interval. Thus, only scan data obtained from a specific depth range are recorded. Induced signals outside the allowed period are not amplified and, thus, are not processed and displayed.

Detector refers to any structure capable of measuring an ultrasound wave or pulse, currently known or developed in the future. Crystals containing dipoles are typically used to measure ultrasound waves. Crystals, such as piezoelectric crystals, shift in dipole orientation in response to an applied electric current. If the applied electric current fluctuates, the crystals vibrate to cause an ultrasound wave in a medium. Conversely, crystals vibrate in response to an ultrasound wave that mechanically deforms the crystals, which changes dipole alignment within the crystal. This, in turn, changes the charge distribution to generate an electric current across a crystal's surface. Electrodes connected to electronic circuitry sense a potential difference across the crystal in relation to the incident mechanical pressure.

Echogenicity refers to the brightness of a tissue in an ultrasound image relative to the adjacent tissues, typically on a B-scan image. Echogenicity is dependent on the amount of ultrasound waves reflected by the tissue. Certain tissues are more echogenic than other tissues. Fatty tissue, for example, is more echogenic than muscle tissue. For identical imaging parameters, fatty tissue will thus appear brighter than muscle tissue. Consequently, image brightness can be used to identify different tissues.

Medical condition refers to a physiological state of a subject, usually a human, that is not normal and would usually benefit from, or require, medical treatment. Such states may arise from a variety of conditions, including diseases, physiological challenges, trauma, infection, stress, drug abuse, and accelerated aging.

Medical treatment refers to an action intended to confer a medical or physiological benefit on a subject, including surgery, catheterization, drug administration (e.g. either by the subject or by a health care worker), exercise, diet and non-invasive medical techniques (e.g. ultrasound).

Plane refers, in a biological context, to the surface of a cross-sectional area of tissue interrogated by an ultrasound probe. In ultrasound, the portion of the tissue included in the measurement or image is more accurately referred to as a volume. The x-dimension of this volume reflects the length of the tissue plane, i.e. the length of imaged tissue. The x-dimension typically varies between 1 and 10 cm or more. The y-dimension reflects tissue depth from the plane, e.g. the distance from the skin surface to a reflection point in the tissue. The y-dimension (or depth of the interrogation) depends, among other things, on the type of transducer, the type of tissue, and the frequency with which the ultrasound beam is transmitted. With higher frequencies, tissue penetration decreases and the maximum depth from the tissue plane will decrease. The y-dimension typically varies between 1 and 30 cm. The z-dimension corresponds to the width of the plane that is interrogated. It typically varies between 1 and 15-20 mm.

Skin refers to the external tissue layer in humans and animals consisting of epidermis and dermis.

Skin Related Definitions:
Epidermis refers to the outer, protective, nonvascular layer of the skin of vertebrates, covering the dermis. The epidermis consists histologically of five layers, i.e. the stratum corneum, the stratum lucidum, the stratum granulosum, the stratum spinosum, and the stratum basale.
Dermis refers to the sensitive connective tissue layer of the skin located below the epidermis, containing nerve endings, sweat and sebaceous glands, and blood and lymph vessels. Histologically, the dermis consists of a papillary layer and a reticular layer. The papillary layer contains the vessels and nerve endings supplying the epidermis. The reticular consists predominantly of elastic fibers and collagen.
Subcutaneous tissue layer refers to a tissue layer located below the skin. This tissue layer is typically characterized by a loose meshwork of connective tissue such as collagen and elastic fibers. It is rich in small vessels, e.g., arterioles and venoles, and capillaries.

Therapeutic agent refers to an active substance that produces a beneficial effect in a subject when administered in a therapeutically effective amount using a therapeutically effective modality. Such agents include active substances directed to specific physiological processes or systems, such as, but not limited to, diuretic, hepatic, pulmonary, vascular, muscular, cardiac or diabetic agents. Usually, such agents will modify the physiological performance of a target tissue or cell in order to shift the physiological performance of the target tissue or cell towards a more homeostatic physiological state. Such agents can be administered in as collection of active substances or therapeutic agents.

Therapeutic kit refers to a collection of components that can be used in a medical treatment.

Therapeutic dosage refers to a dosage considered to be sufficient to produce an intended effect.

Therapeutically effective modality refers to a manner in which a medical treatment is performed and is considered to be sufficient to produce an intended effect.

Tissue refers to an organized biomaterial usually composed of cells.

Transmission frequency refers to the frequency of the ultrasound wave that is being transmitted from the ultrasound source. Transmission frequency typically ranges between 0.2 MHz and 25 MHz. Higher frequencies usually provide higher spatial resolution. Tissue penetration decreases with higher frequencies, especially in dense fat tissue. Lower transmission frequencies are generally characterized by lower spatial resolution with improved tissue penetration. Methods and devices for optimizing and matching transmission frequencies to the measured object's acoustic properties are described herein.

Ultrasound pulse refers to any ultrasound wave transmitted by an ultrasound source. Typically, the pulse will have a predetermined amplitude, frequency, and wave shape. Ultrasound pulses may range in frequency between about 20 kHz and 20 MHz or higher. Preferably, for measurements pulses range from about 2.5 MHz to 25 MHz and more preferably from about 3.5 to 10 MHz. Ultrasound pulses may consist of sine waves with single frequency or varying frequencies, as well as single amplitudes and varying amplitudes. In addition to sine waves, square waves or any other wave pattern may be employed. Square waves may be obtained by adding single-frequency sine waves to other sine waves. The summation of waves can then result in a square wave pattern.

Ultrasound signal refers to any ultrasound wave measured by an ultrasound detector after it has been reflected from the interface of an object or tissue. Ultrasound signals may range in frequency between 20 kHz and 20 Mhz or higher. Preferably, for measurements signals range from 2.5 Mhz to 25 Mhz.

Ultrasound source refers to any structure capable of generating an ultrasound wave or pulse, currently known or developed in the future. Crystals containing dipoles are typically used to generate an ultrasound wave above 20 khz. Crystals, such as piezoelectric crystals, that vibrate in response to an electric current applied to the crystal can be used as an ultrasound source. An ultrasound generator can include single or multiple ultrasound sources that can be arranged at different angles to produce ultrasound beams (or pulses) with variable transmission angles. In some ultrasound generators, multiple ultrasound sources may be arranged in a linear fashion. This arrangement of ultrasound sources is also referred to as a linear array. With linear arrays, ultrasound sources are typically fired sequentially, although simultaneous firing of groups of adjacent ultrasound sources or other firing patterns of individual or groups of ultrasound sources with various time delays can be achieved as described herein or developed in the art. The time delay between individual or group firings can be used to vary the depth of the beam in an object.

Ultrasound or ultrasonic wave refers to either an ultrasound signal or pulse.

2.0 Introduction

The inventors of the present invention recognized, among other things, a need in the ultrasound field for transmission films and holders for transducers, particularly for transducers adapted to interrogate the external surface of an object or a subject. Transmission films and holders of the invention can provide for hygienic probes of tissues, including the genitalia where transmission of sexually transmitted diseases (“STDs”) may occur. Such transmission films and holders can offer many advantages including, reduced transmission of STDs and other contact transmitted diseases, low cost of manufacture, enhanced or more reproducible recording, convenience and hygiene of disposable articles, and reduced operator error in maintaining inter-patient hygiene. The inventors also discovered a need for dispensing such transmission films and holders in a convenient, user and manufacturing friendly manner. Such transmission films and holders can be used with any probe that contacts a subject's skin and passes energy into, or receives energy from, one or more tissues of the subject, particularly ultrasound probes.

The present invention also recognized for the first time that pre-application of an acoustic coupling gel can enhance ultrasound measurements and provide more convenient, hygienic or sterile protection. The invention includes machine or pre-application of an acoustic coupling material to either a transmission film or holder. Previously, it was not recognized that a application of an acoustic coupling material at the manufacturing stage to transmission films or probe holders could improve or make diagnostic ultrasound measurements more reproducible or convenient.

Nor was it recognized that transmission films or holders for ultrasound applications could be rigid. Previous work also failed to recognize that rigid transmission films or holders could be used to alleviate many of the problems associated with using conventional latex condoms as protective drapes around an ultrasound probe, such as condom dragging, condom slippage, condom tearing, cumbersome condom donning, cumbersome condom removal, cumbersome application of acoustic coupling gel to the internal surface of the condom before donning, condom phobia of subjects, reduced integrity of probe hygiene between patients, increased probability of transmission of contact transmitted diseases between patients, excessive acoustic coupling gel application, difficulties in maintaining probe hygiene and cleanliness and condom fit on probes of different sizes (one size condom does not adequately fit all probes).

Section 3 primarily describes various aspects of holders and transmission films of the invention and related systems.

Section 4 includes descriptions of stacks of holders and holder dispensers.

Section 5 includes method for making probe holders and devices related to manufacturing probe holders and any ultrasonically interrogatable surface using acoustic coupling fluids or gels.

By way of introduction, and not limitation of the various embodiments of the invention, the invention includes at least eight general aspects:
1) a device for interrogation of a tissue based on a holder for a probe that transmits or receives energy and is typically adapted for interrogation on an epidermal or epithelial surface and at least one transmission film,
2) a device for interrogation of an object comprising a rigid holder adapted to fit and ultrasonic probe that is typically adapted for interrogation on an epidermal or epithelial surface and where the holder has at least one sonolucent film disposed across an interrogation window,
3) a stack of probe holders, preferably disposable holders, for convenient use or application of transmission enhancing fluids (including gels),
4) a device for interrogation of an object comprising a holder of a generally predetermined shape and three dimensional dimensions adapted to fit a probe without the probe necessarily providing shape to the holder when the probe is inserted into the holder and the holder includes an interrogation region,
5) a device for manufacturing ultrasound related devices or testing surfaces comprising an acoustic coupling gel or fluid dispenser for dispensing on a surface and an ultrasonic detector and source for interrogating the surface,
6) a device for manufacturing ultrasound related devices or testing surfaces comprising an acoustic coupling gel or fluid dispenser for dispensing on a transmission film or holder and a transport system to transfer the transmission film or holder, and
7) interrogation systems and therapeutic kits related to 1 to 6.

These aspects of the invention, as well as others described herein, can be achieved using the methods and devices described herein. To gain a full appreciation of the scope of the invention, it will be further recognized that various aspects of the invention can be combined to make desirable embodiments of the invention. Such combinations can result in particularly useful and robust embodiments of the invention.

3.0 Holders and Transmission Films

Introduction

Previously, patients interrogated with medical probes based on electromagnetic or ultrasonic energy, or objects interrogated by other detection probes that send or receive electromagnetic or ultrasonic energy, were protected from exposure to potentially contagious agents or other medical or environmental contamination hazards by loose fitting drapes. Typically, drapes were slipped over the probe and the open end tied or taped to the probe. Drapes, however, have significant drawbacks that include: 1) difficult to reproduce dragging effects if the probe is moved across a surface, 2) slippage of the drape off the probe, 3) tearing of the drape while moving the probe, 4) cumbersome placement of the drape on the probe, 5) cumbersome removal of the drape from the probe, 6) cumbersome application of transmission enhancement fluids to the drape before or after donning, 7) long installation time of the drape on the probe, 8) potential for reduced integrity of probe hygiene between objects to be interrogated due to the ill fitting nature of many drapes, 9) increased probability of transmission of contact transmitted diseases between patients, and 10) excessive or imprecise application of transmission enhancement fluids. In addition, there are many instances where drapes are not available for probes and probes must be properly cleaned or sterilized before the next use. This can lead to difficulties or inconveniences in maintaining probe hygiene and cleanliness and can subsequently lead to undesirable pathogen transmission between patients or contamination of an object to be contaminated by the object previously interrogated.

Holders with Interrogation Windows and Holders with Sonolucent Films

The present invention provides for devices for protecting probes and patients from unwanted contamination. The invention provides for holders that fit over the probe and insulate the probe from the outside environment. Typically, the holder is pre-sized to fit the probe. The holder comprises a material that minimizes attenuation or interference with the electromagnetic or ultrasonic signal produced or received by the probe.

Preferably, the holder is rigid. A rigid holder offers the advantage of holding the shape of the probe without the probe being introduced into the holder. A rigid holder can allow for rapid engagement of the probe with the holder and easy removal. Rigid holders may also be stacked for quick and reliable deployment, as described herein. In addition, the holders can include a predetermined amount of transmission enhancement fluid or layer that increases the reproducibility of interrogation using probes that can be used with transmission enhancement fluids. Such holders, and other described herein, can also be used with probes designed to interrogate biological and as well as manufactured objects, such as pipes, concrete, tanks with fluid, plastic objects and glass objects.

The invention finds particular application in ultrasonic interrogation from a skin interrogation site. The invention includes a device that comprises a holder for an ultrasonic probe that is adapted for skin-interrogation of tissues subjacent to a skin interrogation site. The holders are typically designed to permit interrogation of a patient by placing the holder, directly or with a gel, on the patient's skin or integument, not mucous membranes. Although, in some embodiments of the invention holders can be designed to contact mucous membranes, such as the vaginal wall or mouth. The holder is adapted to fit at least a portion of the interrogation surface of the ultrasonic probe. The interrogation surface of the probe is a portion of the probe designed to transmit or receive ultrasonic waves. The holder typically includes 1) a securing portion for securing the holder to the ultrasonic probe and 2) an interrogation window in acoustic alignment with at least a section of the interrogation surface. A sonolucent film may be included to cover the interrogation window.

The holder is typically made of a hard, polymeric material. The holder is usually designed to be flexible while maintaining the general shape of the probe for which it was designed to fit. The holder may have flexible extensions or flanges that secure the holder to the probe. The holder may be constructed from a rigid, flexible plastic that can bend slightly as the probe is inserted in the holder. Once the probe is inserted in the holder, the holder will grasp the probe as the flexible plastic will be tensioned on the probe. Typically, the polymeric material from which the holder is made is more flexible than the sonolucent-polymeric material of the sonolucent film. Often the sonolucent film will be made of a different material than the remainder of the holder. This permits the introduction of different materials into the holder so as to select the desired structural properties of the holder, such as for securing the holder to the probe, without necessarily being constrained by the transmission properties of the transmission film which may not have as rigid or robust structural properties as the rest of the holder. The polymeric material of the holder often is a rigid, injection molded polymer, which is easy to manufacture on a large scale. The holder is usually designed with a region(s) having a cross sectional thickness greater than the sonolucent film's cross sectional thickness. By making the sides of the holder thicker than the film greater rigidity can be obtained while minimizing the effect of transmission through the film. The holder is usually made of a polymeric material, which can be referred to as a holder-polymeric material.

The interrogation window is typically an integral part of the holder. The interrogation window permits the passage of waveform energy of type the probe is design to transmit or receive. Often the holder and the interrogation window are one piece. For example the interrogation window is a molded portion of the holder. The interrogation window usually has about the same surface area as the interrogation surface of the ultrasonic probe for which the holder is designed. The holder and interrogation window, as well as the sonolucent film, can be made of one acoustic coupling material, preferably a plastic. The interrogation window is typically about 1 cm² to 10 cm²; preferably 5 cm² or less in surface area or 2 cm² or less in surface area.

The sonolucent film is typically made of a sonolucent-polymeric material. The film typically covers the interrogation window. Sonolucent-polymeric materials are materials that permit the passage of ultrasonic waves, typically at least about 80 to 99 percent passage of the ultrasonic waves hitting the material. However, in some embodiments, reflection of ultrasonic waves can provide an advantage as a marker. The sonolucent film is usually substantially planar interrogation surface of the probe. The interrogation window may also be made of a rigid polymer with a substantially planar surface that holds the sonolucent film. Usually, the sonolucent-polymeric material is more flexible than the holder-polymeric material. The sonolucent film may be heated welded or sealed to the holder. The sonolucent film may be a layer of acoustic coupling material made of a pliable polymer matrix. The sonolucent film may include an applied gel on the sonolucent film's exterior interrogation-side (i.e. the side in contact with the patient) to enhance acoustic communication. The sonolucent film may include an applied gel on the sonolucent film's interior interrogation-side to enhance acoustic communication.

The holder may comprise securing members for securing the holder to the ultrasonic probe. Generally, the securing members are shaped to fit the probe using a friction fit and are located in the securing portion of the holder. The device of the invention can include an ultrasound probe adapted to fit the holder.

The holders of the invention can offer storage and contamination prevention features. A holder can be stored in a container to protect it. The container may be opened just prior to use to permit inserting the probe into the holder. Typically, the holder is made of a molded plastic and contained in a hygienic or sterile container to protect it from contamination prior to use. The holders can also be designed to stack. The invention includes a plurality of the holders wherein each holder has an exterior contour and an interior contour and the exterior contour is designed to fit into the interior contour of the next holder in the stack.

Examples of Holders

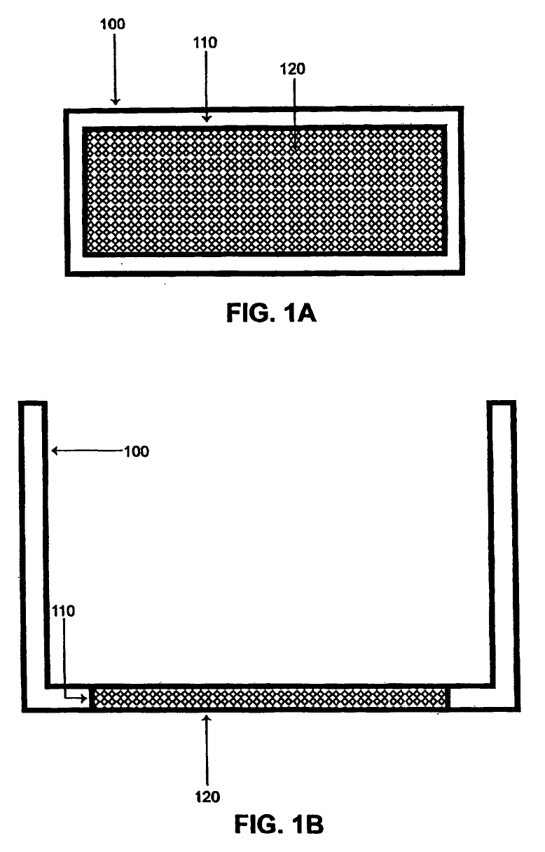

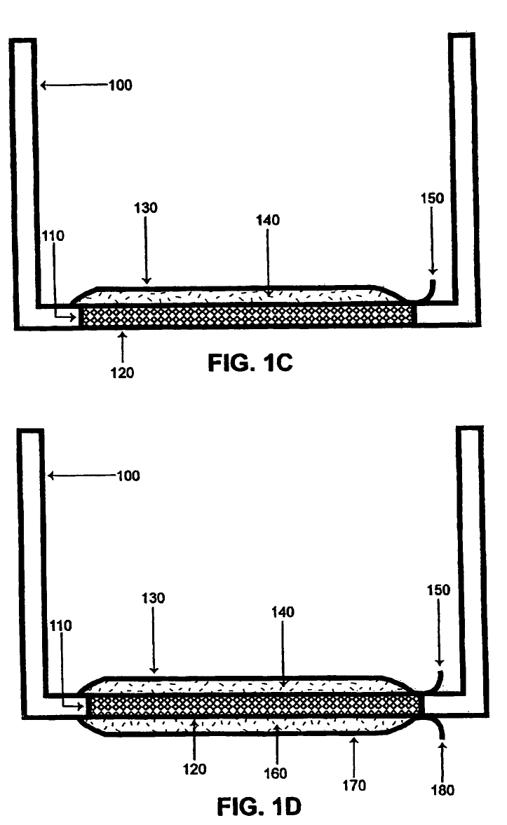

FIG. 1A shows an example of a holder of the invention from a cross sectional view looking towards the exterior of the interrogation surface of the holder. The holder 100 has the over-all shape of an ultrasonic probe to which it is designed to fit. The interrogation window 110 is an integral portion of the holder and is sized to correspond to the area of the ultrasonic detectors or transmitters. Thus, when the holder is placed on the probe the interrogation window 110 substantially aligns with the area of the ultrasonic detectors or transmitters to permit transmission or detection. The holder 100 may include a sonolucent film 120 covering the interrogation window, see hatched area.

FIG. 1B shows an example of a holder of the invention from a cross sectional view with the exterior of the interrogation surface of the holder facing down. The holder 100 has the over-all shape of an ultrasonic probe to which it is designed to fit and may have side that follow the contours of the probe. The thickness of the holder is usually sufficient to maintain rigidity of the holder. The interrogation window 110 forms an opening in this embodiment and its border can have the same thickness as the remainder of the holder. The holder 100 may include a sonolucent film 120 covering the interrogation window. The sonolucent film is shown as having the same thickness as the interrogation window and may be made of the same or different material from the remainder of the holder.

FIG. 1C shows an example of a holder of the invention from a cross sectional view with the exterior of the interrogation surface of the holder facing down. In addition to the features of the holder 100 described in FIGS. 1A and 1B, the holder in FIG. 1C has additional features that provide for more convenient holder deployment on the probe while enhancing hygiene. The interrogation window 110 has a layer of acoustic coupling gel 140 that has been applied in a predetermined amount and which covers the interior interrogation surface of the sonolucent film 120. A protective, removable film 130 covers the acoustic coupling gel 140 to protect it from contamination and evaporation. When the holder is to be used, the operator moves the protective, removable film 130 by grasping the removal tab 150.

FIG. 1D shows an example of a holder of the invention from a cross sectional view with the exterior of the interrogation surface of the holder facing down. In addition to the features of the holder 100 described in FIGS. 1A through 1C, the holder of FIG. 1D has additional features that provide for more convenient holder deployment on the probe while enhancing hygiene. The interrogation window 110 has a second layer of acoustic coupling gel 160 that has been applied in a predetermined amount and which covers the exterior of the interrogation surface of the sonolucent film 120. A second, protective, removable film 170 covers the acoustic coupling gel 160 to protect it from displacement, contamination and evaporation. When the holder is to be used the operator moves the protective, removable film 170 by grasping the removal tab 180. The holder 100 may include a layer of acoustic coupling gel 160 without including a layer of acoustic coupling gel 140.

Holders with Predetermined Shapes

The invention also provides for devices that have shapes that are predetermined in size and designed for a probe or series of probes. The invention includes a device that comprises a rigid, plastic holder for a probe, such as an ultrasound source or detector. The rigid, plastic holder is of a generally predetermined shape. The holder also typically has generally preset three-dimensional dimensions that are maintained without the insertion of the probe, such as an ultrasound source or detector. The rigid, plastic holder comprises an interrogation region for interrogation of an exterior interrogation surface of an object or patient. The interrogation region can be dimensioned to snugly fit over a housing or frame for the probe's electromagnetic or ultrasound source or detector while permitting interrogation through the interrogation region. The interrogation region engages with the ultrasound source or detector housing or frame using mechanisms described herein, or developed in the art now or in the future. The holder can include an ultrasound probe mechanically compatible with the rigid, plastic holder, and optionally includes a system for interrogation, signal processing and conveyance of interrogation information. To provide sufficient rigidity, holders typically have sides that are between 0.5 and 4 mm in thickness, and preferably, 0.75 mm and 2 mm in thickness. Some embodiments, however, may have thinner or thicker sides. Where a portion of the holder is expected to pass waveform energy, the thickness of such portion can be generally thinner (usually about 25 to 200 percent thinner than the sides) such as less than about 1 mm. Such thinner section can improve the ability to pass waveform energy. The holders of the invention can be sized for probes that typically comprise a medical probe selected from the group consisting of a MRI probe, an ultrasound probe, a radioactivity probe and a photon probe.

Molded Holders

In one embodiment it will advantageous to manufacture holders of the invention in a molded fashion to reduce cost per item while maintaining a quality product. The invention includes a molded device, comprising a rigid, plastic holder for an electromagnetic wave or ultrasound source (or detector or both). The rigid, plastic holder is of a generally predetermined shape and three-dimensional dimensions without an inserted probe. For example, the holder, without the insertion of the probe, is able to receive the probe without the need for holding the open end of the holder open. Typically, the holder has sufficient structural integrity to generally maintain a probe shape, in the x, y, and z dimensions, to accommodate the probe to be inserted. The rigid, plastic holder includes an interrogation region for interrogation of an exterior interrogation surface. The interrogation region is dimensioned to snugly fit over a housing of the probe while permitting interrogation through the interrogation region and the interrogation region engages with the housing. The device can be injection molded. The device can include a machine applied acoustic gel layer, or transmission enhancement fluid or gel layer, on the interrogation region to facilitate acoustic coupling between the interrogation region and an ultrasound source or detector. The holder can include a cap that snugly fits over the interrogation region intended to be in contact with the object or patient.

Removable and Disposable Holders

In another embodiment, the invention provides for devices that comprise a removable holder for an ultrasound probe, the removable holder comprising a proximal region for interrogation of an external interrogation surface. The proximal region is adapted for acoustic alignment with an ultrasound source or detector. The proximal region includes an interrogation surface that permits interrogation with an ultrasound probe. The holder includes a distal region that is slidably engagable with the ultrasound probe while maintaining the acoustic alignment. Typically, the proximal region is molded and the distal region may be molded as well. Such devices can also be used with probes that transmit or receive electromagnetic energy. One of the most significant advantages of the present invention is that in most embodiments the holders may be donned and removed with an operator using only one hand or by only handling the probe and with the necessity of handling the holder itself.

The distal region is designed to hold the probe or its housing. This can be accomplished by using a rigid plastic material in the distal region. The plastic can be selected so that the finished product is rigid while possessing sufficient flexibility to “snap” the probe into the distal region. Typically, the distal region conforms substantially to the shape of at least the widest portion of the probe. The device distal region can include friction engagable nibs to grasp the probe. The friction engagable nibs can include an entry angle that is mechanically compatible with a friction engagable depression or depressions on the probe. In such cases it will be desirable to provide a probe with predetermined friction engagable depression or depressions that physically correspond to, or mate with, the friction engagable nibs of the holder. Alternatively, the holder may have depressions and the probe may have the nibs. Friction engagable depressions can include an entry angle that is mechanically compatible with a friction engagable nib. The distal region can also include a probe engager to engage the probe, such as for the universal holders described herein.

The distal region can be made of many different types of materials described herein, or developed in the art now or in the future. The distal region or the holder may be made of materials selected from the group consisting of polycarbonates, polystyrenes, polyethylenes, polyvinyl chlorides, polypropylenes, and cyclo-olefins (including copolymers). Other types of polymers may also be used and can be selected on the basis of rigidity, ease of manufacture, cost, ability to pass ultrasonic or electromagnetic waves and the degree of flexibility. Preferably, the distal region is made of a rigid polymer.

The interrogation surface is often a film that passes ultrasonic waves or can acoustically couple the probe to the interrogation site. Films that can be used are any films that permit the passage of ultrasonic waves described herein, or developed in the art now or in the future. The films desirably also prevent or reduce the passage or transmission of pathogens or contaminants (such as toxins or toxic substances) to the probe. Candidate films can be easily tested for their ultrasonic properties and selected based on their ability to be compatible with the desired type of interrogation. Generally films are selected based on their ability to pass ultrasonic waves, the amount of interference, amount of echogencity, flexibility, cost, ultrasonic attenuation, biocompatibility and manufacturing requirements. In some embodiments the film is more rigid than a polyurethane film of about 2 mil, and made of a polymer that passes at least 50 percent of ultrasonic waves reaching the film's surface, preferably at least 90 percent, and more preferably at least 95 percent. The film can be rigid or pliable. Films can also be used to make substantially all of the device from one material. In some embodiments it will be desirable to use features of other embodiments described herein. For example, in some embodiments it will be desirable that a portion, or substantially all, of the film maintains a substantially planar surface without insertion of a probe into the holder.

The interrogation surface may include an interior surface with an acoustic coupling gel of a known volume. The known volume is usually selected based on a sufficient volume to permit acoustic contact with the interrogation surface of the probe once it is inserted into the holder. If the holder is to be used with different sized probes, preferably the volume is sufficient to accommodate such probes while maintaining acoustic contact.

The interrogation surface can be made of an acoustic coupling material selected from the group consisting of polyethylenes, polymethylpentenes, polyurethanes, cyclo-olefins, cyclo-olefin copolymers, and polypropylenes. The interrogation surface can be made of carbon-based polymers, silicon based polymers, latex and other easily extruded or manufactured materials. Such materials should be selected as a barrier to prevent transmission of agents, pathogens or other harmful or contaminating substances to the probe. In another embodiment, holders of the invention can include a transmission enhancing fluid or gel to improve interrogation with a probe.

In another embodiment of the invention, holders may also be designed to fit more than one size of probe. Such holders can be termed “universal holders” since they can fit probes of different sizes. However, in most instances such holders will be designed to fit probes within a prescribed size range. Such holders can be designed to include a distal region that comprises a contractible and expandable sizing element to grasp the probe. Typically the contractible and expandable sizing element is made of an elastomeric material. Such holders can individually and separately accommodate an ultrasound probe selected from a collection of ultrasound probes of different volumes, preferably such volumes are about fifty percent of the volume of the probe with the largest volume.

In another embodiment of the invention, holders may also be designed with an applicator or dispenser to apply or dispense a transmission enhancing fluid. Such embodiments of the invention offer the advantages of 1) providing more accurate dispensing of such fluids compared to manual dispensing from a squirt bottle, 2) single hand operation of the probe and application of the gel and 3) less risk of contamination between objects or patients because the applicator can be disposable. Such holders can be termed “dispensing holders” that can allow a probe to interrogate and permit application of fluids, such as gels. Preferably, the holder comprises a reservoir with at least one orifice for allowing the fluid to exit and a pressure device that applies pressure directly or indirectly to the fluid to cause the fluid to controllably exit the orifice. For instance, a mechanical plunger that is controlled by an electric motor or piston can be used to push gel out of the reservoir. The probe can include a switch for controlling the amount of gel to be applied. Dispensing means known in the art or developed in the future may also be used. The reservoir can be designed to be disposable to reduce contamination between interrogation of different objects. The reservoir can be adapted to fit a reusable plunger so that the reservoir can be replaced without the necessity of replacing the plunger. Alternatively, the reservoir may be manually compressed to dispense a gel.

Examples of Universal Holders and Holders with Applicators

This subsection describes examples of holders that can be used with probes, particularly medical probes, such as ultrasound probes.

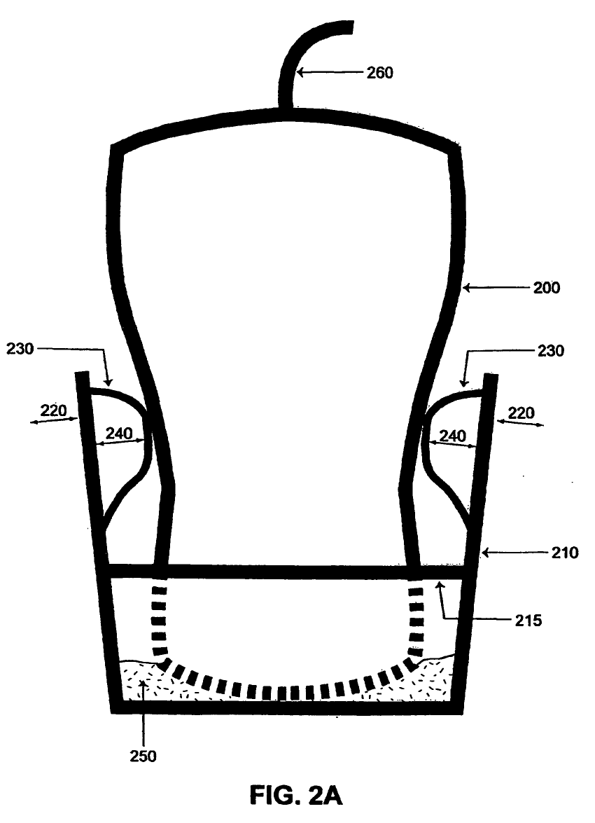

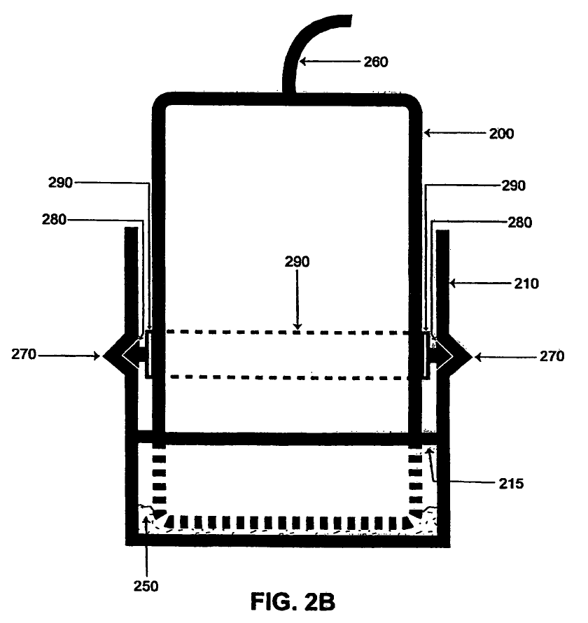

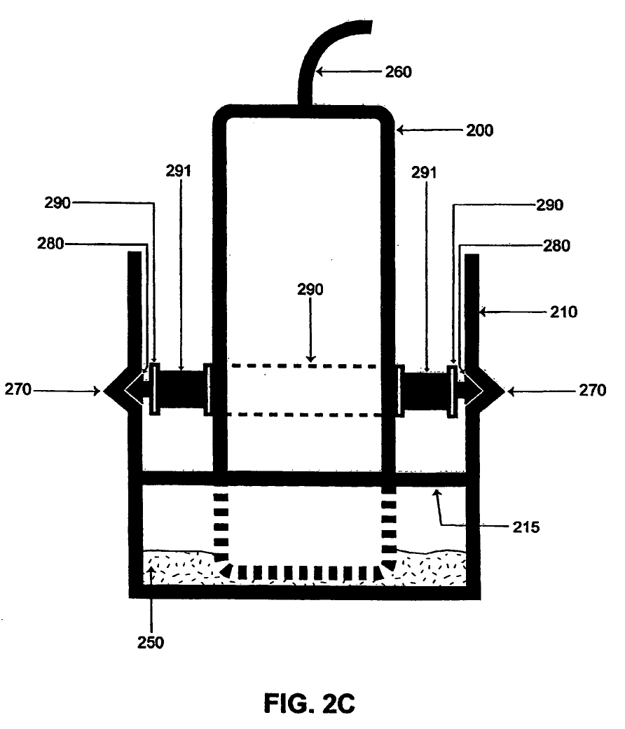

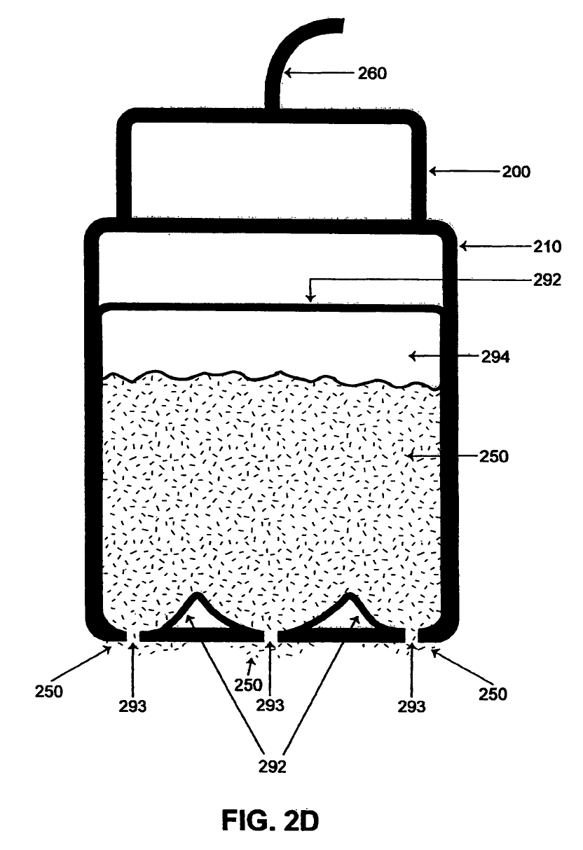

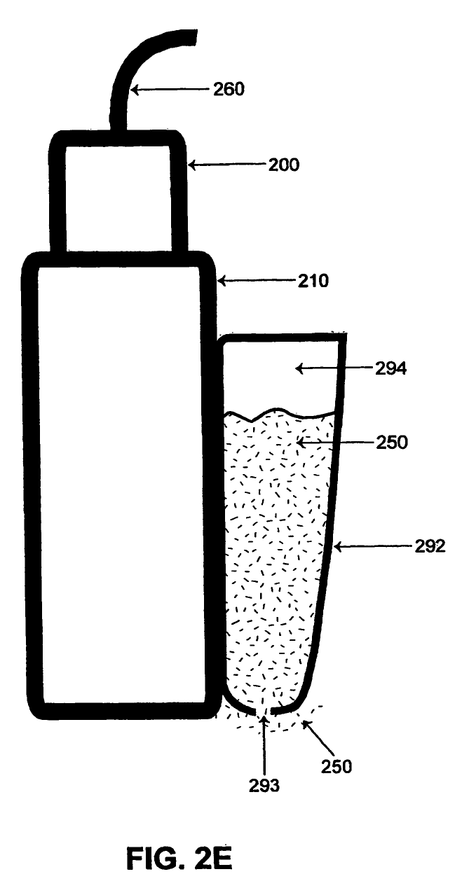

FIG. 2A shows an example of a holder of the invention from a front view with a probe. The holder 210 has dimensions to fit different sizes of probes to which it is designed to fit. The probe 200 with a connection 260 is inserted into the holder 210 and sides of the holder can be flexed outward 220. Compressible and expandable members 230, which are usually made of an elastomeric material and can be of variable length on the inside of the holder, can compress and expand 240 to fit the dimension and contours of the inserted probe. The side 215 of the holder 210 can extend up from the base of the holder but does not need to extend completely to the distal end of the holder. Such sides can be used in other holders described herein and can vary in length as desired for a particular application. The height of such sides can be selected to minimize contamination of the probe. The holder 210 may include a predetermined amount of acoustic coupling gel 250 at the base or the proximal end of the holder for ultrasonic probe, see stippled area.

FIG. 2B shows an example of a holder of the invention from a front view with a probe and a collar 290. The holder 210 has dimensions to fit different sizes of probes to which it is designed to fit. The probe 200 with a connection 260 is inserted into the holder 210, which has engagement sites 270 to secure the engagement sites 280 on the collar 290. The side 215 of the holder 210 can extend up from the base of the holder but does not need to extend completely to the distal end of the holder. Such sides can be used in other holders described herein and can vary in length as desired for a particular application. The height of such sides can be selected to minimize contamination of the probe. The holder 210 may include a predetermined amount of acoustic coupling gel 250 at the base or the proximal end of the holder for an ultrasonic probe, see stippled area.

FIG. 2C shows an example of a holder of the invention from a front view with a smaller probe compared to FIG. 2B and a collar 290. The holder 210 has dimensions to fit different sizes of probes to which it is designed to fit. The probe 200 with a connection 260 is inserted into the holder 210, which has engagement sites 270 to secure the engagement sites 280 on the collar 290. The extended member 291 of the collar 290 has attached engagement sites 280 and permits the holder 210 to be used with different sized probes. The side 215 of the holder 210 can extend up from the base of the holder. The holder 210 may include a predetermined amount of acoustic coupling gel 250 at the base or the proximal end of the holder for an ultrasonic probe, see stippled area.

FIG. 2D shows an example of a holder of the invention from a front view with a probe and an applicator system. The holder 210 is dimensioned to fit a probe. The probe 200 with a connection 260 is inserted into the holder 210, which has a reservoir 292 of acoustic coupling gel 250 that can be mechanically or manually squeezed or pushed out of the reservoir 292 at application sites 293. The base of reservoir 292 may not necessarily be completely flush with the base of the holder, as it may be a distendable bag filled with gel. The reservoir may have an empty portion 294. To apply more gel the operator may squeeze the reservoir or activate a plunger or other dispensing mechanism to increase the pressure in the reservoir and force the gel out of it.

FIG. 2E shows an example of a holder of the invention from a side view with a probe and an applicator system. The holder 210 is dimensioned to fit a probe. The probe 200 with a connection 260 is inserted into the holder 210, which has a reservoir 292 of acoustic coupling gel 250 that can be mechanically or manually squeezed or pushed out of the reservoir 292 at application sites 293. The base of reservoir 292 may not necessarily be completely flush with the base of the holder, as it may be a distendable bag filled with gel. To apply more gel the operator may squeeze the reservoir or activate a plunger or other dispensing mechanism to increase the pressure in the reservoir and force the gel out of it. Such reservoirs of the invention offer the advantage of permitting operation and dispensation of a gel with a single hand.

Examples of Holders for Ultrasound Transducers

This subsection describes additional examples of holders that can be used with probes, particularly medical probes, such as ultrasound probes.

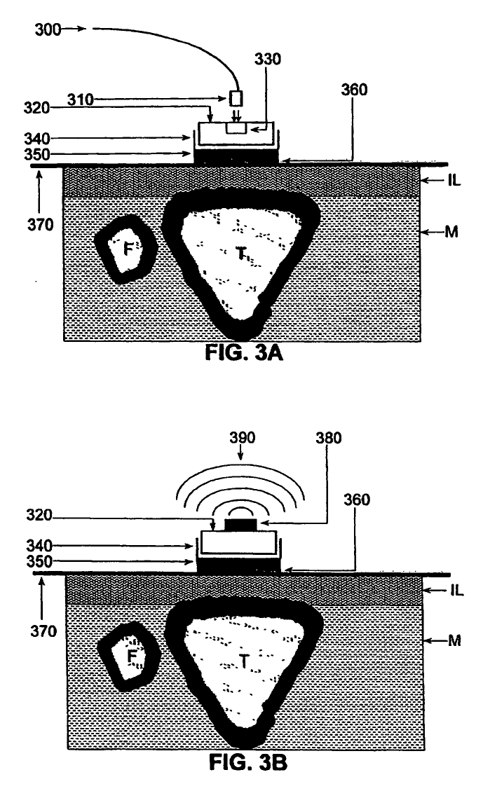

FIGS. 3A and B show embodiments of the invention comprising an ultrasound transducer secured to a subject or a tissue surface with an adhesive probe holder, which is preferably used for intermittent or continuous recording. The ultrasound transducer can be electrically coupled to an ultrasound computational unit (not shown) using a lightweight wire 300. An electrical connector 310 connects the computational unit and the ultrasound transducer 320 using an electrical connecting socket or connector means 330. The ultrasound transducer 320 is optionally seated inside a positioning frame 340. The undersurface of the positioning frame consists of an acoustic coupler 350. The positioning frame is attached to the subject or tissue surface using an adhesive 360. The adhesive 360 can acoustically couple the ultrasound probe to the skin of the subject or the interrogated tissue surface 370. The adhesive 360 can also be interspersed with an acoustic coupling material, such as a gel (not shown). Tibia is “T”. Fibula is “F”. Muscle is “M” and interstitial layer is “IL”. FIG. 3B shows that the ultrasound transducer 320 can also be coupled to an ultrasound computational unit (not shown) using an infrared coupler or a radio frequency coupler 380 or other connector means that transmits signals 390 to an ultrasound computational unit.

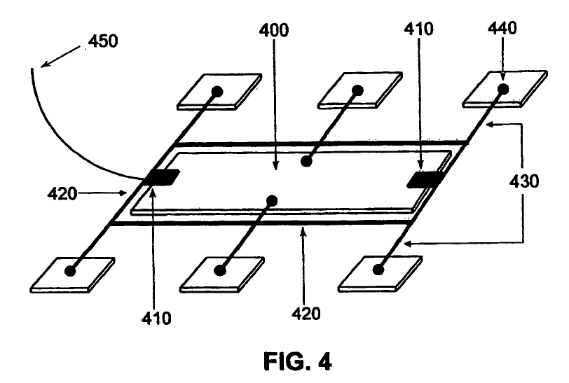

FIG. 4 shows a holder with a frame 420 that can have extending members 440 that can be secured to the skin and away from the interrogation site in order to reduce artifacts associated with probe placement. The structure of the frame can resemble a spider, where the body of the frame 420 secures the micro-transducer 400 and the legs of the positioning frame 430 secure the frame to the skin application site. Such spider embodiments of the positioning frame are particularly useful for securing the micro-transducer to an appendage region either by taping the legs or adjusting the legs to interlock. The positioning frame may be disposable and optionally include a sterile film disposed in the frame so as to provide a sterile micro-transducer surface. Acoustic coupling materials can be applied to either side of the film to enhance acoustic communication. The positioning frame can also include other fastening systems known in the art, such as Velcro. Alternatively the micro-transducer can be secured with adhesive coating. The adhesive coating can be applied to the skin of the subject or as part of the micro-transducer. Preferably, when acoustic coupling materials are applied to the skin, such as a gel, an adhesive can be included in the acoustic coupling materials to secure the micro-transducer.

In another embodiment the ultrasound probe holder is adapted to attach to a securing member that secures an appendage of the human and secures the ultrasound probe holder. This embodiment can immobilize the appendage and/or the micro-transducer. The acoustical coupling material can be secured in acoustical contact with the surface of the skin. An acoustic coupling gel can be optionally applied between the surface of the skin and the acoustical coupling material.

4.0 Stacks of Holders and Dispensers for Holders

Stacks and Advantages of Stacks

The invention provides for the first time stacks of holders for probes that either transmit or receive waveform energy. The invention provides for a device comprising a stack of holders for a probe. Each holder comprises an exterior region and an interior region. The exterior region of each holder is adapted to fit into the interior region of the next holder in the stack. Alternatively, the interior region of each holder is adapted to fit into the exterior region of the next holder in the stack. As another alternative, the exterior region of each holder is adapted to fit into the interior region of the next holder in the stack and the interior region each holder is adapted to fit into the exterior region of the next holder in the stack. The holders can be any of the holders described herein and with the appropriate design modifications, if necessary.

Preferably, each holder in the stack has an exterior interrogation surface. If the stack is a stack of ultrasound probe holders, the stack can include a plurality of acoustic coupling gel exterior layers. Each acoustic coupling gel exterior layer can comprise a machine applied, predetermined volume of acoustic coupling gel on a plurality of the exterior interrogation surfaces of some or all of the holders.

Preferably, each holder in the stack has an interior interrogation surface. If the stack is a stack of ultrasound probe holders, the stack can include a plurality of acoustic coupling gel interior layers. Each acoustic coupling gel interior layer can comprise a machine applied, predetermined volume of acoustic coupling gel on a plurality of the interior interrogation surfaces of some or all of the holders. Some stacks may have holders with both an exterior and an interior layer.

The stack can further comprise a plurality of removable films in contact with the acoustic coupling gel exterior or interior layers. The removable films help prevent contamination of the exterior layer.

Preferably, each holder has an exterior interrogation surface and is adapted to fit a cap. Each cap is adapted to fit and sized to the exterior interrogation surface or probe. Each cap may be also adapter to fit the interior region of a holder to permit nested stacking of capped holders. A stack may include a plurality of holders with a plurality of caps.

Preferably, each cap further comprises a machine applied, predetermined volume of acoustic coupling gel applied to either the exterior or interior surface of the holder. Preferably, the acoustic coupling gel is in acoustic contact with the interior interrogation surface of each holder. Preferably, each cap includes a hydrophobic surface in contact with the acoustic coupling gel. The hydrophobic surface helps prevent the acoustic coupling gel from adhering to the cap. Typically, the holders and caps are made of molded plastic and may differ in the material from which they are molded.

In one embodiment, the invention provides for a stack-dispensing device to facilitate the removal of holders from a stack. A stack-dispensing device includes a surface to raise a stack within a rack Alternatively, the side of the rack may be lowered to allow easy access to the uppermost holder in the stack. Typically, the stack will have five to ten holders and the rack will extend to the distal portion of the uppermost holder. The stack can be raised by a spring or piston mechanism. Such lifting mechanism preferably has enough resistance to a downward force to permit donning of the holder to the probe, thereby allowing the operator to insert the probe into the holder without lowering the stack to a position that would significantly interfere with donning the holder.

Stacks of the invention offer a number of advantages, including 1) one handed donning of holders on to probes, 2) convenient maintenance of the hygiene or sterility of holders, 3) convenient storage of holders, and 4) easy repetitive donning of holders on to probes for rapid multiple interrogations.

Examples of Stacks

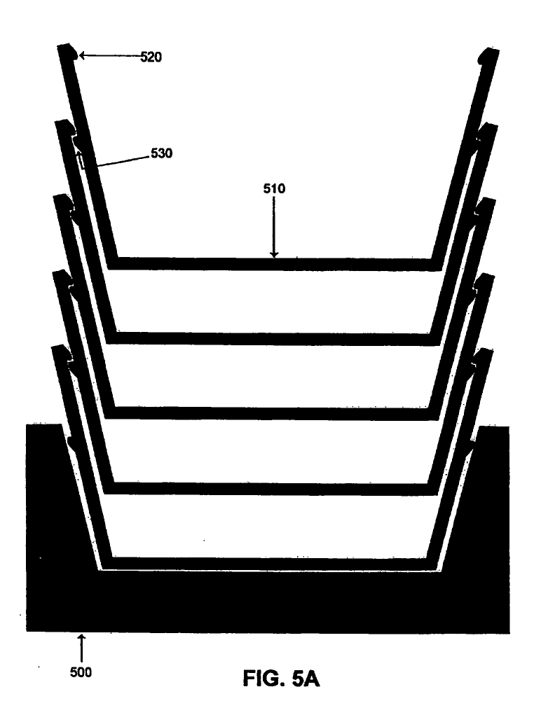

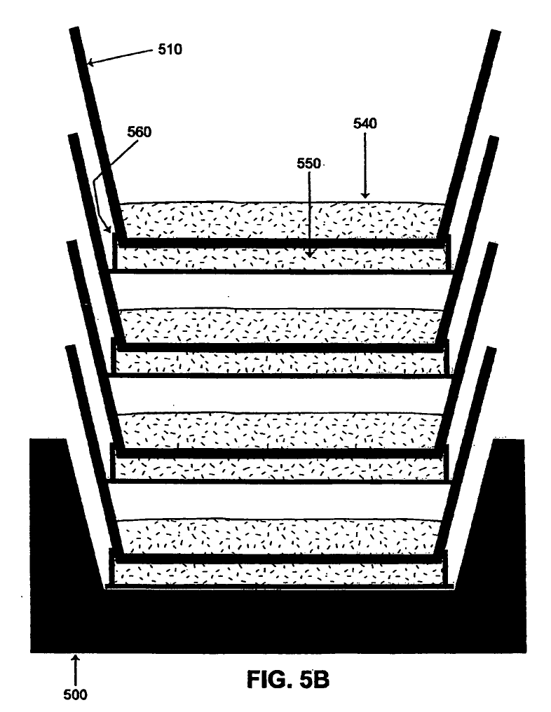

FIG. 5A shows an example of a stack of holders of the invention in a cross sectional view with a rack 500. Holders 510 are dimensioned to fit inside of each other, which can create a nested stack of holders. Each holder 510 is preferably designed with flexible sides to allow inserting of holders into each other. Each holder 510 is preferably designed with an inner nib 520. The inner nib 520 can be designed to secure the holder to another holder or a probe or both. The inner nib can be designed to removably, inter-lock with an outer nib 530. The outer nib 530 can serve to removably secure holders together by interlocking with an inner nib 520. Flexible sides of the holder faciliate inserting a holder into another holder. The sides may engaged to be slightly bent inward for insertion. Once the holder is inserted into another holder the sides may be released and the sides spring out to engage the holder into which it was inserted. To remove a holder, the sides may be engaged to be slightly bent inward so that the inner nib and outer nib are disengaged to permit removal of holder. An operator for instance can bend the sides to permit removal of a holder. This can be accomplished with the probe inserted into the holder to be removed and bending the sides inward to permit release of such holder from the stack. Holders may be designed and sized so that the proximal region or end of a holder will contact the inner surface of the holder into which it is inserted. The dimensions of the proximal region will limit how far the holder can be inserted into each other, which can be adjusted to reduce or increase the overall dimension of the nested stack.

FIG. 5B shows an example of a stack of holders with caps of the invention in a cross sectional view with a rack 500. Holders 510 and caps 560 are dimensioned to fit inside of each other and the combination thereof can be inserted into a holder, which can create a nested stack of holders. Each holder 510 is preferably designed to engage a cap 560. Each holder 510 is preferably manufactured with an inner layer of transmission enhancing fluid 540. The inner layer of transmission enhancing fluid 540 can be dispensed in a sterile or hygienic fashion and contact with an insert cap can be avoided by dimensioning the cap to engage a holder before touching such layer. Each holder 510 can also be manufactured with a cap 560 with an outer layer of transmission enhancing fluid 550. If a cap 560 is manufactured, an outer layer of transmission enhancing fluid 550 the cap may be designed with a hydrophobic material(s) to repel an outer layer of transmission enhancing fluid 550 and to allow removal of the cap without having a major portion of the outer layer of transmission enhancing fluid 550 stick to the cap. The stack can be placed in a rack 500 to maintain or organize the stack.

Example of a Holder Dispensing Station

FIG. 5C shows an example of a stack of holders in a rack with a platen of the invention in a cross sectional view. Holders 510 and caps 560 are dimensioned to fit inside of each other and the combination thereof can be insert into a holder, which can create a nested stack of holders. Each holder 510 is preferably designed to engage a cap 560. A platen 570 can be used to support a stack and a member 580 can be used to elevate or lower a stack. As each holder is used, the stack is preferably raised by increasing the dimension 590. This facilitates removal of a holder from the rack by permitting easier access to the upper most holder at the top of the rack 500. Each holder 510 is preferably manufactured with an inner layer of transmission enhancing fluid 540. Each holder 510 can also be manufactured with a cap 560 with an outer layer of transmission enhancing fluid 550.

5.0 Devices for Manufacture and Methods

The invention includes methods and devices for manufacturing and testing articles of the invention. Such methods and devices can also be used for manufacturing and testing many other types of objects, particular objects that can have a structural feature interrogated by ultrasonic methods.

The invention includes a method for manufacturing holders and films of the invention. The method can include a molding process to make the entire holder, including any transmission area as an integral unit. Alternatively, the transmission area may be a window with no material. A film can then be affixed to the holder by heat welding, pressure welding, adhesives (including solvent adhesives), radio frequency welding or a combination of welding techniques. Any other applicable bonding or welding techniques known in the art or developed in the future can be used as well. During the welding process it can be useful to examine the Vicar temperature of the film to reduce holidays and other inconsistencies in a weld. Preferably, plastics are used to make the holder.

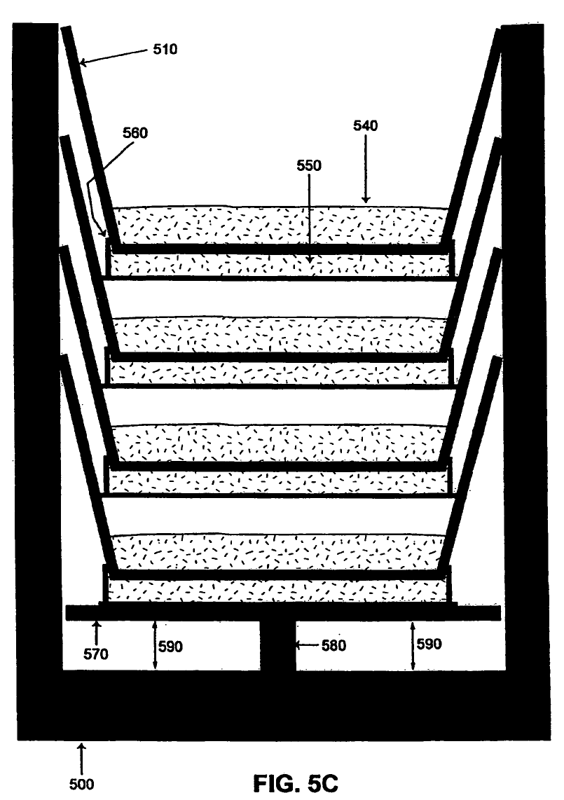

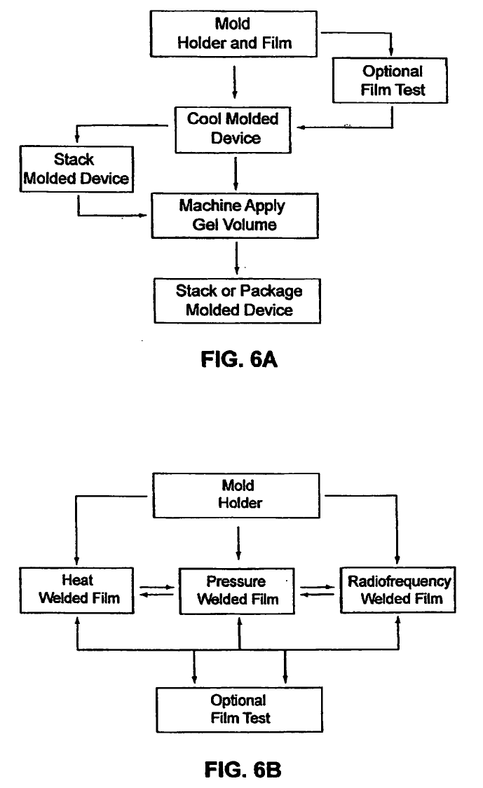

FIG. 6A shows an example of a manufacturing process of the invention as a flow chart. The holder can be molded as described herein. The holder can then be optionally tested. For instance, the holder's transmission abilities can be tested as described herein or its structural integrity as described herein. The manufactured device can be cooled and then tested as well. Once the device is sufficiently cool, typically when the chance of deformation is low, the device can be stacked and optionally gel applied, such as acoustic coupling gel. Alternatively, the gel can be applied prior to stacking. The stack can then be packaged if so desired. Preferably, the final steps process are hygienic or sterile or the stack is sterilized (e.g. electron beam or UV methods) after or just prior to packaging.

FIG. 6B shows an example of a manufacturing process of the invention as a flow chart. The holder can be molded as described herein with an open transmission window. A film can than be applied to the holder. Preferably, heat welding, pressure welding, radio frequency welding, or a combination thereof can be used to affix the film to the holder. The holder can then be optionally tested. The method described in FIG. 6A can be readily combined with such techniques.

The methods described herein can be readily constructed as a series of instructions in a computer program. Such programs can be used to control equipment to automate such processes. In addition such computer programs can be designed to utilize data from the manufacturing process to adjust the manufacturing process. Such computer programs can be stored on a computer readable medium, such as a disk, hard drive or magnetic material based storage system.

Typically, if welded films are employed to form a device of the invention, the average Shore A hardness will be greater than about 50 to about 90. The tensile stress may between at least about 800 and 3,000 psi or greater.

Other materials which are useful for forming articles of the invention include films based on elastomeric materials, as well as flexible non-elastomeric materials such as nylons, polyethylene terephthalate, and olefinic homopolymers and copolymers, e.g., ultra-low density polyethylene. As used herein, the term “elastomeric” in reference to thermoplastic materials useful for forming articles in accordance with the present invention, means a material which subsequent to elongation thereof under an applied tensional force, regains at least a significant portion of its original dimensional characteristics when the applied tensional force is released.

Illustrative of thermoplastic elastomeric materials which may find utility in the broad practice of the present invention are: polyurethane materials, as for example the polyester-based polyurethane material commercially available from Mobay Corporation (Plastics and Rubber Division, Pittsburgh, Pa.) under the trademark TEXIN.RTM., and the thermoplastic polyurethane elastomers which are commercially available from BASF Corporation (Parsippany, N.J.) under the trademark ELASTOLLAN.RTM.; polyester elastomer, such as the block copolymers of polybutylene terephthalate and long-chain polyether glycols, which are available commercially from E.I. Du Pont de Nemours and Company, Inc. (Polymer Products Department, Engineering Polymers Division, Wilmington, Del.) under the trademark HYTREL.RTM.; polyether blockamides, such as those commercially available from Atochem, Inc. (Glennrock, N.J.) under the trademark PEBAX.RTM.; multiblock rubber-based copolymers, particularly those in which the rubber block component is based on butadiene, isoprene, or ethylene/butylene, such as those commercially available from Dow Chemical Company (Midland, Mich.) under the trademark ATTANE.RTM.; as well as any other suitable homopolymers and copolymers, and mixtures, alloys, and composites thereof.

In addition, multiblock rubber-based copolymers may be employed as materials of construction for articles of the present invention may be varied widely, it being understood that the non-rubber repeating units of the copolymer may be derived from any suitable monomer(s), as for example, (meth)acrylate esters, such as methyl methacrylate, cyclohexylmethacrylate, etc.; vinyl arylenes, such as styrene; etc. Illustrative multiblock butadiene-based copolymers which may be usefully employed in the broad practice of the present invention include those variously described in U.S. Pat. Nos. 3,297,793; 3,595,942; 3,402,159; 3,842,029; and 3,694,523, the disclosures of which hereby are incorporated by reference herein. Various multiblock styrene-containing polymers may be usefully employed to form the articles of the present invention. Examples of this type of polymer are triblock styrene-butadiene-styrene copolymers and styrene-ethylene/butylene-styrene terpolymers commercially available under the trademark KRATON from Shell Chemical Company (Houston, Tex.). Other examples of small block butadiene-styrene copolymers commercialized by Firestone Synthetic Rubber & Latex Company (Akron, Ohio) are marketed under the trademark STEREON.

Suitable materials for the invention can be selected by examining the shear stiffness, and tensile energy value, as well as other measurements of stiffness or rigidity.

The shear stiffness value of the films can be determined by applying opposing parallel forces to the film by a KES-FB1 tensile-shear tester, of the type described at pages 34-36 of The Standardization and Analysis of Hand Evaluation, Second Edition, 1980, by S. Kawabata. These opposing parallel forces are applied until a maximum offset angle of 8.degree. is reached. A tension load of 5 grams force per centimeter (gf/cm) is applied to the specimen for such shear testing, yielding a shear stiffness value as a measure of the conformability of the film material. Numerically, the lower the shear stiffness, sometimes denoted hereinafter as G, the more conformable the film material. The shear stiffness value has units of gf/cm degree.

The tensile energy value of the films can be performed on the KES-FB1 tensile-shear tester, by the procedure described at pages 28-30 of the Kawabata text identified above. The tensile energy value measures the stress/strain character of the material at a maximum load of 50 gf/cm. Due to the excessive “stretchiness” of some the film material, a sample length of 2.5 centimeters is used in such tensile test. The units of the tensile energy value are gf/cm/cm.sup.2. The tensile energy is the area under stress/strain curve, and it relates to the energy which is absorbed by the polymer under a specified stress (50 gf/cm). Generally, the more energy the polymer can absorb, the more extensible it is. Thus, higher tensile energy values are associated with higher extensibility of the film. Preferably, films and holders are rigid, comparable in rigidity at least to polypropylene cartons for consumable liquids (about 5 to 1 mm in thickness) and more preferably comparable in rigidity at least to polypropylene microtiter plates (about 1.5 to 2.5 mm in thickness).

The invention includes devices for manufacturing ultrasound-related devices or other articles of manufacture or objects that may require ultrasonic testing. Such manufacturing or testing devices of the invention are particularly suitable to an automation and mass production process where the throughput of the process is 10,000 samples a day or higher. One such testing device comprises an acoustic coupling fluid dispenser to dispense a selected volume of an acoustic coupling fluid on an acoustically transmissible solid substrate, such as the object to be tested. The acoustic coupling fluid dispenser comprises an orifice or channel in liquid communication with a reservoir. The acoustic coupling fluid is emitted from the orifice and can be computer controlled.

The testing device can include a transfer system to transfer the acoustically transmissible solid substrate to a predetermined location in geometric register with the dispenser or its orifice. This permits the orifice to emit the acoustic coupling fluid onto the acoustically transmissible solid substrate in a desired fashion. An x, y positioner can be used to align the dispenser if necessary. Such, positioners for predetermined X, Y coordinates, can be made using lead screws having an accurate and fine pitch with stepper motors (e.g., Compumotor Stages from Parker, Rohnert Park, Calif., USA). The device can include an ultrasound detection system to detect the distribution of the acoustic coupling fluid onto the acoustically transmissible solid substrate. The device can also have a second acoustic coupling fluid dispenser to dispense a selected volume of an acoustic coupling fluid on additional acoustically transmissible solid substrates. The second acoustic coupling fluid dispenser comprises a second orifice in liquid communication with the reservoir. The acoustic coupling fluid is emitted from the second orifice. The first dispenser can be used for testing and the second dispenser can deliver a predetermined amount of gel for future use. Preferably, the device is designed with an acoustic coupling fluid dispenser that can dispense a gel.

The device can include a computational unit to manage workflow to the acoustic coupling fluid dispenser through the transfer system. The transfer system can be a Shuttleworth conveyor-based system (e.g. slip-torque conveyor system by Shuttleworth, Ind., USA). The device can include an acoustic coupling fluid dispenser and the transfer system can process at least about 1,000 acoustically transmissible solid substrates per hour, preferably at least 5,000 to 10,000 samples per hour (about 3 samples per second), and more preferably at least 10,000 to 50,000 samples per hour (about 15 samples per second or about 1 sample every 70 milliseconds). Higher rates of throughput can be achieved by parallel processing using multiples transfer lanes and multiple dispensers. High rates of dispensing can be achieve through the use of solenoid valves, particular electronically controlled valves and a relatively high pressure fluid channel or reservoir. The device can also be designed with an acoustic coupling fluid dispenser that can dispense a volatile acoustic coupling liquid, such as isopropyl alcohol, ethanol, methanol or acetone. In certain applications this offers a distinct advantage because the article or object can be tested and the testing fluid is easily removed through evaporation. It can also concurrently form the function of cleaning or sterilizing the article or object.

In another embodiment, the invention includes a device for dispensing transmission-enhancing fluids. Such devices can be used to manufacture articles of the invention. For example, such a device can be used to manufacture ultrasound related devices (including probes and acoustic coupling surfaces) or for ultrasonically testing surfaces. The device comprises an acoustic coupling fluid dispenser to dispense a predetermined amount (preferably in an automated fashion) of acoustic coupling fluid on a surface with a subjacent layer or layers and a transport system. In another embodiment, the device comprises an acoustic coupling fluid dispenser to dispense a predetermined amount (preferably in an automated fashion) of acoustic coupling fluid on a surface with a subjacent layer or layers, an ultrasound source, and an ultrasound detector located to receive ultrasound waves from the ultrasound source that are transmitted through the surface or reflected from the surface. The device, as with other devices described herein, is useful for testing surfaces for ultrasonic properties (including echnogenicity, BUA, SOS, acoustic impedance, reflectance, transmission, images, reflective distances and phase shifts). The device can include a transfer system to transfer the surface to and from the acoustic coupling fluid dispenser. Preferably, the transfer system is a conveyer based system and the ultrasound source is located to transmit the ultrasound waves though a plane of the transfer system to the ultrasound detector. Usually, the device is constructed so that the ultrasound detector can detect ultrasound signals from substantially all of the surface. The device can include a computational unit that instructs the ultrasound source and detector, as well as other components. The computational unit can be designed to determine whether a structural abnormality exists in the surface based on the ultrasonic reading it receives.

Preferably, the detector is adapted to measure ultrasound signals that have been transmitted through the surface. The computational unit can be designed to estimate or determine one or more of the following ultrasonic properties of the surface or the layer or the layers: 1) BUA, 2) SOS, 3) reflective distance, echogenicity, percent transmission, percent transmission as a function of location of the surface and amplitude analysis. The device can be used to test or make the surfaces of ultrasound probe holders. The surface to be tested is typically part of one or more of the following structures: 1) a sealed compartment containing a fluid, 2) a film that in the absence of an abnormality permits passage of at least about 75% of ultrasonic waves at a frequency of between about 0.1 and 30 MHz, 3) a film with a layer of acoustic coupling liquid, or 4) a liquid in a container.

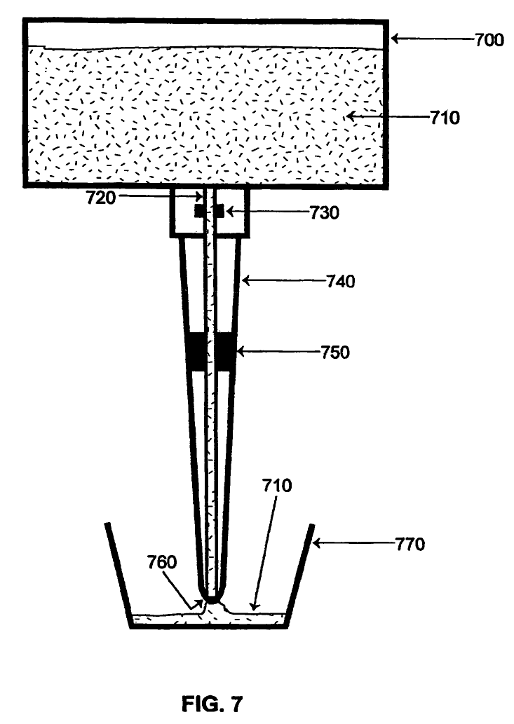

FIG. 7 shows an example of a manufacturing or testing device of the invention for dispensing a transmission enhancing fluid or testing a surface. The device includes a reservoir 700 that contains a transmission enhancing fluid 710. A channel 720 in fluid communication with the reservoir 700 has valve 730 that controllably regulates the amount of fluid dispensed. A housing 740 can encase the channel. The device may optionally include an ultrasound source 750 to aid in the detection of the extent of the dispensation of the fluid or the ultrasonic property of a layer(s). The position of the valve 730 may also be switched with the ultrasonic source 750. The channel 720 leads to an orifice 760 that can emit the fluid. A holder 770 can be placed to receive the fluid that can be dispensed in a predetermined amount.

6.0 Therapeutic Kits and Methods

Kits

The invention also includes therapeutic kits based on the devices and methods of the invention. For example, a therapeutic kit can include an interrogation device described herein, including a holder, probe or interrogation system and a health care product in at least one dosage or a medical treatment. The interrogation device can assist in monitoring for a therapeutic effect of said at least one dosage. The health care product can be designed to produce water loss. The health care product is can be a drug selected from the group consisting of antiarrhythmics, anticholinergics, antihypertensives, alpha- and beta-adrenergic blockers, calcium channel blockers, cardiac glycosides, hydantoin derivatives, and nitrates. The health care product can be a drug selected from the group consisting of diuretics such as aldosterone antagonists, carbonic anhydrase inhibitors, loop diuretics and thiazides or thiazide-like agents.

Use in Medical Conditions and Treatments

The invention can be used in a variety of medical treatments and diagnostics. Often medical treatments are designed to modulate the function of an organ or physiological process. There are numerous examples of treatments that the invention can be used with, such as drugs designed to modulate heart, renal or pulmonary function or improve fluid homeostasis. Methods and devices of the invention can assist in measuring the effectiveness of medical treatments.

Routine periodic examinations, such as part of an annual examination, can monitor long term changes in the physiology due a number of medical conditions, such as those described herein. Such periodic examinations can be applied to the devices and methods described herein.

Examinations during a clinically relevant time period can be used to monitor the progress of expected changes in a subject's physiology. Clinically relevant time periods usually relate to a medical treatment regime or medical conditions. Typical drugs amenable to monitoring include cardiovascular agents and renal agents. Other drugs include anti-hypertensives, diuretics, anticoagulants, and vasoactive substances.

The invention can also be used with surgical treatments. Examples of such surgical treatments include cardiac surgery (e.g., cardiac valve replacement and coronary bypass graft surgery), renal surgery (e.g., surgical or interventional radiologic repair of renal artery stenosis or urinary outflow stenosis), renal and hepatic transplantation, pulmonary arterial embolectomy, peripheral venous or arterial embolectomy, and peripheral vascular surgical and interventional radiologic procedures (e.g., stripping of varicose veins, sclerotherapy, bypass grafting, and thrombolytic therapy), as well as others known in the art or developed in the future. Usually, the clinically relevant time period for monitoring of the efficacy of surgical treatments will be periodically over about days to months.

In other indications related to surgical treatments, monitoring of the side-effects of surgical treatments will be desired. Side effects of surgical treatments include blood loss, cardiac arrest, fat and air embolism, heart failure, hepatic failure, hepatic or renal ischemia and infarction, hypoxic tissue damage, intestinal ischemia and infarction, mechanical tissue damage, myocardial ischemia or infarction, myolysis, pulmonary edema, pulmonary embolism, renal failure, urinary obstruction, respiratory arrest, sepsis, shock, spinal cord injury, over-hydration or dehydration, fluid retention in dependent anatomical regions, lower or upper extremity venous thrombosis, and arterial dissection and/or occlusion.

Another common clinical setting to assess is the efficacy or side-effects of a medical treatment comprising general anesthetic procedures and treatments. Usually, the clinically relevant time period will be during a general anesthetic procedure or treatment and periodically over about 24 to 72 hours post procedure or treatment. Preferably, baseline monitoring prior to general anesthetic procedure or treatment is also conducted. Side-effects of general anesthetic procedures or treatments include hypoxic or embolic brain damage, cardiac arrest, drug-induced complications, heart failure, hypoxic tissue damage, intestinal ischemia and infarction, myocardial ischemia or infarction, myolysis, pulmonary edema, pulmonary embolism, renal failure, respiratory arrest, line sepsis, shock, over-hydration or dehydration, and lower or upper extremity arterial or venous thrombosis.

Intubation of a subject is another common clinical setting to assess the efficacy or side-effects associated with this medical treatment. Usually, the clinically relevant time period will be during an intubation procedure and periodically over about 24 to 72 hours post procedure or treatment. Preferably, baseline monitoring prior to an intubation procedure is also conducted. Side effects of intubation procedures include airway obstruction, airway damage, barotrauma, gastric intubation, tracheal or bronchial perforation, tracheopleural and bronchopleural fistula, tracheoesophageal fistula, hepatic or renal ischemia and infarction, hypoxic brain damage, hypoxic tissue damage, intestinal ischemia and infarction, myocardial ischemia or infarction, pulmonary edema, respiratory arrest, spinal cord and cervical spine injury, and tetraparesis or paraparesis.

### EXAMPLES

General Materials and Methods:

The following materials and methods are exemplary of the materials and methods that can be used to achieve the results described herein. One skilled in the art will readily recognize substitute materials and methods.

In vitro and in vivo ultrasound measurements were performed using an Ultramark 9 HDI ultrasound system (Advanced Technologies Laboratories (“ATL”), 22100 Bothell Everett Hwy, Bothell, Wash. 98041-3003). All examinations were performed using a 5 MHz linear array transducer manufactured by ATL. An acoustic coupling gel was applied to the transducer surface and the object to be examined in order to reduce the impedance mismatch between the transducer surface and the object surface, usually skin. Data were acquired in B-scan mode. Two-dimensional gray-scale images of the various tissue/edema layers were obtained. Images were displayed on a computer monitor attached to the scanner hardware and capable of displaying the full gray scale range. Distance measurements were performed by saving a representative image displaying the various tissue layers, e.g. skin, subcutaneous fat and bone, on the display monitor. A trained physician identified the various tissue interfaces visually and placed cursors manually at the probe/skin, soft-tissue/bone, and other interfaces. Software provided with the ultrasound scanner was then used to calculate the distance between the calipers.

To maintain the anatomic location of the selected sites, a dye was used to mark the sites on the skin of the human subjects. Similarly, in the in vitro experiments, a dye was used to mark the measurement site on the external tissue surface.

### Example 1

### Ultrasonographic Measurement Using Polymer Films

In order to evaluate the accuracy of ultrasonographic measurements with polymers, experiments were performed with different polymer films and an examination tissue. Ultrasonographic measurements were performed in a large piece of muscle tissue obtained from the gluteal region of a pig. The tissue was cut into thin sections using a rotating electric blade.

Two polymer films were tested, Saran Wrap and a metallic impregnated polymer film. Interrogation of the tissue was performed in the presence of acoustic coupling gel applied to both sides of the film. Both films permitted sufficient transmission to record images of the interrogated tissue that were similar to the images obtained in the absence of either film and in the presence of the acoustic coupling gel applied directly to the transducer. The use of the Saran Wrap film, which was attached to the wrap in a drape fashion, however, proved to be surprisingly cumbersome for operating the probe due to its flexible nature. When the probe was moved over the tissue for sweeping type interrogation maneuvers the Saran Wrap film would often move in relation to the probe and require manual adjustment or additional attention by the operator to prevent the film from sliding in relation to the probe. The present invention overcomes many of these difficulties as described herein. For instance, the present invention offers the advantage of providing stable attachment to the probe and/or a rigid interrogation surface.

### PUBLICATIONS

U.S. PATENT DOCUMENTS

| | | |
|---|---|---|
| 4,658,827 | Dec. 21, 1987 | He, P., et al. |
| 4,446,737 | May 8, 1984 | Hottier, F. |
| 4,920,966 | May 1, 1990 | Hon, E. H. |
| 4,224,829 | Sep. 30, 1980 | Kawabuchi, M., et al. |
| 08/731,821 | Filed Oct. 21, 1996 | Lang, P., et al. |
| 4,242,911 | Jan. 6, 1981 | Martin, H. E. |
| 4,688,428 | Aug. 25, 1987 | Nicolas, J.-M. |
| 4,702,258 | Oct. 27, 1987 | Nicolas, J.-M., et al. |
| 4,043,181 | Aug. 23, 1977 | Nigam, A. K. |
| 4,830,015 | May 16, 1989 | Okazaki, K. |
| 5,271,403 | Dec. 21, 1993 | Paulos, J. J. |
| 4,833,323 | May 23, 1989 | Scholze, C. |
| 5,303,708 | Apr. 19, 1994 | Stouffer, J. R. |
| 5,316,003 | May 31, 1994 | Stouffer, J. R. |
| 5,617,864 | Apr. 8, 1997 | Stouffer, J. R., et al. |

FOREIGN PATENT DOCUMENTS

| | | |
|---|---|---|
| PCT WO 93/12419 | Jun. 24, 1993 | Lake, R. J., et al: |

### OTHER PUBLICATIONS

Physician's 1991 drug handbook, pp. 1099-1103, 1991.
Bates, B., et al., in: “A guide to physical examination and history taking, 6th edition”, Bates, B., et al., eds., pp. 427-447, 1995.
Bhagat, P. K., et al., Ultrasound Med Biol, vol. 6, pp. 369-375, 1980.
Braunwald, E., in: “Harrison's Principles of Internal Medicine”, Isselbacher, K. J., Braunwald, E., et al., eds., pp. 183-187, 1994.
Bushberg, J. T., et al. “The essential physics of medical imaging”, 1994.
Carpenter, D. A., et al., Radiology, vol. 195 (2), pp. 563-567, 1995.
Cohen, J. S., et al., Arthritis & Rheumatism, vol. 27, pp. C65, 1984.
Ciocon, J. O., et al., Angiology, vol. 46, pp. 19-25, 1995.
Escoffier, C., et al., Bioeng Skin, vol. 2, pp. 87-94, 1986.
Fornage, B., et al., J Clin Ultrasound, vol. 14, pp. 619-622, 1986.
Fornage, B., et al., Radiology, vol. 189, pp. 69-76, 1993.
Fornage, B. D., Radiologia Medica, vol. 85 (5 Suppl. 1), pp. 149-155, 1993.
Fornage, B. D., Clinics in Diagnostic Ultrasound, vol. 30, pp. 85-98, 1995.
Gottlieb, S. H., in: “Principles of ambulatory medicine”, Barker, L. R., et al., eds., pp. 736-754, 1991.
Goss, S. A., et al., J Acoust Soc Am, vol. 64 (2), pp. 423-457, 1978.
Hermann, R. C., et al., Skin Pharmacol, vol. 1, pp. 128-136, 1988.
Killewich, L. A., et al., Archives of Surgery, vol. 120 (4), pp. 424-426, 1985.
Krijnen, R. M. A., et al., Dermatology, vol. 194, pp. 121-126, 1997.
Ludwig, M., et al., Schweizerische Rundschau fuer Medizin Praxis, vol. 78 (37), pp. 987-992, 1989.
Milner, S. M., et al., Dermatologic Surgery, vol. 23 (1), pp. 43-35, 1997.
Munson, P. L. “Principles of pharmacology. Basic concepts and clinical applications”, 1995.
Querleux, B., et al., Dermatologica, vol. 177, pp. 332-337, 1988.
Reali, U., et al., Plast Reconstr Surg, vol. 93, pp. 1050-1055, 1994.
Richard, S., et al., J Invest Dermatol, vol. 100 (5), pp. 705-709, 1993.
Tan, C. Y., et al., in: “Bioengineering and the skin”, Marks, R., et al., eds., pp. 215-225, 1981.
Williams, P., et al. “Gray's anatomy, 36th British Edition”, 1980.

All documents and publications, including patents and patent application documents, are herein incorporated by reference to the same extent as if each publication were individually incorporated by reference, including U.S. patent application Ser. No. 08/914,527, filed Aug. 19, 1997 by the inventors of the present application.

What is claimed is:

1. A device, comprising a stack of holders for ultrasonic probes, each holder comprises an exterior region and an interior region, and 1) said exterior region is adapted to fit into said interior region, 2) said interior region is adapted to fit into said exterior region or 3) said exterior region is adapted to fit into said interior region and said interior region is adapted to fit into said exterior region.

2. The device of claim 1, wherein each said holder has an exterior interrogation surface and said device further comprises a plurality of acoustic coupling gel exterior layers, each exterior layer comprises a machine applied, predetermined volume of acoustic coupling gel on a plurality of said exterior interrogation surfaces.

3. The device of claim 1, wherein each said holder has an interior interrogation surface and said device further comprises a plurality of acoustic coupling gel interior layers, each interior layer comprises a machine applied, predetermined volume of acoustic coupling gel on a plurality of said interior interrogation surfaces.

4. The device of claim 3, wherein each said holder has an exterior interrogation surface and said device further comprises a plurality of acoustic coupling gel exterior layers, each exterior layer comprises a machine applied, predetermined volume of acoustic coupling gel on a plurality of said exterior interrogation surfaces.

5. The device of claim 4, further comprising a plurality of removable films in contact with said acoustic coupling gel exterior layer, wherein said removable films help prevent contamination of said exterior layer.

6. The device of claim 1, wherein each said holder has an exterior interrogation surface and said device further comprises a plurality of caps, each cap is adapted to fit and sized to said exterior interrogation surface.

7. The device of claim 6, wherein each cap further comprises a machine applied, predetermined volume of acoustic coupling gel and said acoustic coupling gel is in acoustic contact with said exterior interrogation surfaces.

8. The device of claim 7, wherein each cap further comprises a machine applied, predetermined volume of acoustic coupling gel and said acoustic coupling gel is in acoustic contact with said exterior interrogation surfaces.

9. The device of claim 8, wherein each cap further comprises a hydrophobic surface in contact with said acoustic coupling gel, wherein said hydrophobic surface helps prevent said acoustic coupling gel from adhering to said cap.

10. The device of claim 7, wherein said holders and caps are a molded plastic.

11. A therapeutic kit, comprising:
a) a rigid, plastic holder for an ultrasound source or detector, said rigid, plastic holder is of a generally predetermined shape and three dimensional dimensions without an inserted ultrasound source or detector, said rigid, plastic holder comprises an interrogation region for interrogation of an exterior interrogation surface, said interrogation region is dimensioned to snugly fit over a housing or frame for said ultrasound source or detector while permitting interrogation through said interrogation region and said interrogation region engages with said ultrasound source or detector housing or frame, and
b) a health care product in at least one dosage or a medical treatment; wherein said interrogation device can assist in monitoring a therapeutic effect of said at least one dosage.

12. The therapeutic kit of claim 11, wherein said health care product produces water loss.

13. The therapeutic kit of claim 11, wherein said health care product is a drug selected from the group consisting of antiarrhythmics, anticholinergics, antihypertensives, alpha- and beta-adrenergic blockers, calcium channel blockers, cardiac glycosides, hydantoin derivatives, and nitrates.

14. The therapeutic kit of claim 11, wherein said health care product is a drug selected from the group consisting of diuretics such as aldosteron antagonists, carbonic anhydrase inhibitors, loop diuretics and thiazides or thiazide-like agents.

15. A device, comprising a rigid, plastic holder for an ultrasound source or detector, said rigid, plastic holder is of a generally predetermined shape and three dimensional dimensions without an inserted ultrasound source or detector, said rigid, plastic holder comprises an interrogation region for interrogation of an exterior interrogation surface, said interrogation region is dimensioned to snugly fit over a housing or frame for said ultrasound source or detector while permitting interrogation through said interrogation region and said interrogation region engages with said ultrasound source or detector housing or frame, wherein the holder configured to be compatible with a plurality of differently sized ultrasonic probes.

16. The device of claim 15, wherein said ultrasound source or detector is adapted for in situ ultrasound measurements.

17. The device of claim 16, wherein said rigid, plastic holder is adapted for securing an acoustic coupling material to a surface of an object or subject for in situ ultrasound measurements.

18. The device of claim 17, wherein said acoustic coupling material has an adhesive coating or adhesive properties.
